(19) United States
(12) Patent Application Publication
(10) Pub. No.: US 2026/0078449 A1
(43) Pub. Date: Mar. 19, 2026

(54) Methods and Reagents for Determining Distance Recurrence and Overall Survival in Subjects Afflicted with Breast Cancer

(71) Applicant: Delphi Diagnostics, Inc., Houston, TX (US)

(72) Inventors: Drew Watson, Los Altos, CA (US); Federico A. Monzon, Houston, TX (US); Condie E. Carmack, Missouri City, TX (US)

(21) Appl. No.: 19/327,272

(22) Filed: Sep. 12, 2025

Related U.S. Application Data
(60) Provisional application No. 63/694,280, filed on Sep. 13, 2024.

(51) Int. Cl. C12Q 1/6886

(52) U.S. Cl. CPC C12Q 1/6886; C12Q 2600/118; C12Q 2600/158

(57) ABSTRACT

The present disclosure relates generally to methods and systems for determining cancer recurrence risk. More specifically, the disclosure relates to methods and systems for determining breast cancer recurrence risk using gene expression analysis of multiple biomarkers.

### TECHNICAL FIELD

The present disclosure relates generally to methods and systems for determining cancer occurrence or recurrence risk. More specifically, the disclosure relates to methods and systems for determining breast cancer occurrence or recurrence risk using gene expression analysis of multiple biomarkers.

### SUMMARY

Unless otherwise defined, all technical and scientific terms used herein have the same meaning as commonly understood by one of ordinary skills in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The present disclosure provides methods and systems for determining a risk of recurrence or occurrence of breast cancer in a subject. In some aspects, the methods may involve applying a hazard regression analysis to select metrics, including a recurrence score (RS) and an endocrine activity index score (EAI). The RS may be determined by detected amounts of mRNA expressed from certain genes, including estrogen receptor 1 (ESR1), progesterone receptor (PGR), BCL2, and SCUBE2 genes. The EAI may be determined by detected amounts of mRNA expressed from a plurality of genes, which may include SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KCNE4 genes. In some instances, additional histological metrics may be included in the recurrence score, e.g., a histological tumor grading, e.g., mitotic count, nuclear grade, H-score, or another, but this may not always be required.

In some embodiments, the methods may involve determining mRNA expression levels of a panel of biomarkers comprising at least 19 biomarkers selected from a group of genes. The methods may further involve identifying a risk of breast cancer in the subject by applying a multivariate proportional hazards regression model to the outputs of the mRNA expression levels of the panel of biomarkers.

The disclosure also provides reaction mixtures and kits comprising sets of nucleic acid probes for determining mRNA expression levels of panels of biomarkers relevant to breast cancer risk assessment.

These aspects and other features and advantages of the invention are described below in more detail.

### BRIEF DESCRIPTION OF THE FIGURES

The foregoing and other features and advantages of the present invention will be more fully understood from the following detailed description of illustrative embodiments taken in conjunction with the accompanying drawings in which:

FIG. 1 illustrates an exemplary method for determining breast cancer recurrence risk using gene expression analysis.

FIG. 2 is a Kaplan-Meier Curves for distant recurrence-free interval (DFS) Stratified by Recurrence Score (RS) and SET2,3 Risk Groups.

It should be understood that the drawings and pictures are not necessarily to scale.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Terminology

Unless otherwise defined, all technical and scientific terms used herein have the same meaning as commonly understood by one of ordinary skills in the art to which this invention belongs. Methods and materials are described herein for use in the present invention; other, suitable methods and materials known in the art can also be used. The materials, methods, and examples are illustrative only and not intended to be limiting.

As used herein, “about” and the term “approximately,” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself. Thus, by way of example, “about 10” should be understood to mean “10” and a range no larger than “9-11”. For clarity, as used herein, designation of a range of values includes all integers within or defining the range, and all subranges defined by integers within the range.

As used herein, the term “and/or” refers to and encompasses any and all possible combinations of one or more of the associated listed items, as well as the lack of combinations when interpreted in the alternative (“or”).

As used herein, term “or” refers to any one member of a particular list and also includes any combination of members of that list.

The singular forms of the articles “a,” “an,” and “the” include plural references unless the context clearly dictates otherwise. For example, the term “a pest” or “at least one pest” can include a plurality of proteins, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation. For example, where the disclosure describes a gene in a list, e.g., Ki-67 without the other genes in the list, e.g., ESR1, PGR, BCL2, STK₁₅, Survivin, Cyclin B₁, MYBL₂, Stromelysis 3 Cathepsin L₂, HER₂, GRB₇, SLC39A6, STC2, CA12, PDZK1, NPY1R, CD2, MAPT, QDPR, AZGP1, ABAT, ADCY1, CD3D, NAT1, MRPS30, DNAJC12, SCUBE2, and KCNE4 genes, this is also intended to provide antecedent basis for a negative limitation.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Prognosis” refers to a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable survival following cancer treatments, such as a conventional cancer therapy.

“Sequence identity” or “identity” in the context of two polynucleotides sequences makes reference to the residues in the two sequences that are the same when aligned for maximum correspondence over a specified comparison window.

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, the term “homology” as used herein often pertains to sequence identity, e.g., probe homology to a target sequence. Probes relative to a target mRNA sequence, e.g., sequence of a probe targeting ESR1, PGR, BCL2, STK₁₅, Survivin, Ki-67, Cyclin B₁, MYBL₂, Stromelysis 3 Cathepsin L₂, HER₂, GRB₇, SLC39A6, STC2, CA12, PDZK1, NPY1R, CD2, MAPT, QDPR, AZGP1, ABAT, ADCY1, CD3D, NAT1, MRPS30, DNAJC12, SCUBE2, and KCNE4 genes. Sequence identity is the amount of characters which match exactly between two different sequences. Hereby, gaps are not counted and the measurement is relational to the shorter of the two sequences. This has the effect that sequence identity is not transitive, i.e. if sequence A=B and B=C then A is not necessarily equal C (in terms of the identity distance measure): A: AAGGCT, B: AAGGC, C: AAGGCAT/Here identity(A,B)=100% (5 identical nucleotides/min(length(A),length(B))). Identity(B,C)=100%, but identity(A,C)=85% ((6 identical nucleotides/7)). So 100% identity does not mean two sequences are the same. Sequence similarity is first of all a general description of a relationship but nevertheless its more or less common practice to define similarity as an optimal matching problem (for sequence alignments or unless defined otherwise). Hereby, the optimal matching algorithm finds the minimal number of edit operations (inserts, deletes, and substitutions) in order to transform the one sequence into an exact copy of the other sequence being aligned (edit distance). Using this, the percentage sequence similarity of the examples above are sim (A,B)=60%, sim (B,C)=60%, sim (A,C)=86% (semi-global, sim=1−(edit distance/unaligned length of the shorter sequence)).

The terms “hormonal” and “endocrine” therapy or treatment are used interchangeably herein to refer to an agent which blocks the body's ability to produce a specific hormone (e.g., estrogen) or interferes with hormone action.

The term “determining an expression level” as used herein means the application of a gene specific reagent such as a probe, primer or antibody and/or a method to a sample, for example a sample of the subject and/or a control sample, for ascertaining or measuring quantitatively, semi-quantitatively or qualitatively the amount of a gene or genes, for example the amount of mRNA. For example, a level of a gene can be determined by a number of methods including for example immunoassays including for example hybridization (e.g., bDNA hybridization), immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR, serial analysis of gene expression (SAGE), Northern Blot, digital molecular barcoding technology, for example Nanostring: nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene® ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The term “sample” as used herein includes any biological specimen obtained from a patient. Samples include, without limitation, breast tissue biopsies, whole blood, plasma, serum, red blood cells, white blood cells (e.g., from breast tissue lymph), ductal lavage fluid, nipple aspirate, lymph (e.g., disseminated tumor cells of the lymph node), fine needle aspirate (e.g., harvested by fine needle aspiration that is directed to a target, such as a tumor, a tissue sample (e.g., tumor tissue) such as a biopsy of a tumor (e.g., needle biopsy of breast tissue) or a lymph node (e.g., sentinel lymph node biopsy), and cellular extracts thereof. In some embodiments, the sample is a formalin fixed paraffin embedded (FFPE) tumor tissue sample, e.g., from a solid tumor of the breast.

A “biopsy” refers to the process of removing a tissue sample for diagnostic or prognostic evaluation, and to the tissue specimen itself. Any biopsy technique known in the art can be applied to the methods and compositions of the present invention. The biopsy technique applied will generally depend on the tissue type to be evaluated and the size and type of the tumor (i.e., solid or suspended (i.e., blood or ascites)), among other factors. Representative biopsy techniques include excisional biopsy, incisional biopsy, needle biopsy (e.g., core needle biopsy, fine-needle aspiration biopsy, etc.), surgical biopsy, and bone marrow biopsy. Biopsy techniques are discussed, for example, in Harrison's Principles of Internal Medicine, Kasper, et al., eds., 16th ed., 2005, Chapter 70, and throughout Part V. One skilled in the art will appreciate that biopsy techniques can be performed to identify cancerous and/or precancerous cells in a given tissue sample.

Statistical significance refers to the claim that a result from data generated through experimentation is likely to be attributable to a specific cause. A high degree of statistical significance indicates that an observed relationship is unlikely to be due to chance. Significance tests use methods of inference to establish statistical significance and support or reject claims based on observed data. Every significance test begins with a null hypothesis, designated as H₀, that represents the precise claim to be proved. Often the null hypothesis is a statement of no difference. The claim about the population that evidence is being sought for is the alternative hypothesis, designated as Hₐ. The calculation of statistical significance is subject to a certain degree of error. In significance testing, the pre-specified p-value is the probability of obtaining test results at least as extreme as the result actually observed under the assumption that the null hypothesis is true. In significance testing, p-values less than 0.05 and 0.01 are often pre-specified to establish statistical significance.

### DETAILED DESCRIPTION

### I. Overview

All of the functionalities described in connection with one embodiment of the methods, compositions, or formulations described herein are intended to be applicable to the additional embodiments of the methods, compositions, or formulations described herein except where expressly stated or where the feature or function is incompatible with the additional embodiments. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

Breast cancer is a systemic disease and is treated using multimodality therapy. Surgery and radiation therapy are used for local control of the disease, and subsequently, the majority of breast cancer patients are also offered some form of systemic therapy. Currently, almost all patients with estrogen receptor (ER) positive tumors are offered hormonal therapy, and patients with ER-negative tumors are generally offered chemotherapy. A small percentage of patients with ER+ tumors are also treated with chemotherapy. The tumor size, lymph node status, and the overall receptor profile are critical in deciding the type of systemic therapy. Although patients with multiple positive nodes and higher-stage disease (regardless of receptor status) often receive chemotherapy, the type of systemic therapy offered to patients with lymph node-negative or early-stage disease is often based on receptor profile. Statistically, about ˜70% of breast cancers are ER+/HER2-negative and almost all such patients are offered hormonal therapy, but only a small percentage receive and benefit from additional chemotherapy. The analysis and clinical trials involved in determining the prognosis of such ER+/HER2-negative breast cancer patients, as well as which ER+/HER2-negative breast cancer patients will benefit from receiving chemotherapy, is a significant undertaking in molecular testing in breast cancer.

Several tests have been evaluated in trials, including Oncotype DX (ODX)™ ODX™ is also known as the 21 gene assay (16 cancer-related and 5 housekeeping genes) and was designed to estimate the risk of distant recurrence for patients with ER-positive, lymph node-negative breast cancers when treated with endocrine therapy, specifically tamoxifen. The test is reported as a numerical “recurrence score (RS)” which is calculated using a formula based on gene expression levels of the 16 cancer-related genes and ranges from 0 to 100. Based on earlier studies that utilized tissue blocks from National Surgical Adjuvant Breast and Bowel Project B-14 and B-20 clinical trials, ODX RS categories were defined as low-risk (score 0 to <18, average risk of 7% assuming patient receives tamoxifen for 5 y), intermediate-risk (score 18 to 30, average risk 14%), and high-risk (≥31, average risk approaching 30%). These retrospective studies showed the benefit of chemotherapy only in the high-risk group with no benefit in low-risk and negligible benefit in the intermediate-risk group. The test became commercially available in the later part of 2004 and has become the standard of care. The Trial Assigning IndividuaLized Options for Treatment was designed to assess the usefulness of ODX testing, which arbitrarily redefined the intermediate-risk group as score 11 to 25. Consequently, patients with scores 0 to 10 received only endocrine therapy, and patients with scores >25 received both endocrine and chemotherapy. Patients with ODX RS 11 to 25 were randomized to receive either endocrine therapy alone or both endocrine and chemotherapy. After 9 years of average follow-up, the recurrence rate and survival were similar between the endocrine-only group and the chemo-endocrine group, concluding that there is a lack of chemotherapy benefit in patients with RS 11 to 25. Since then, the new cut-off for chemotherapy consideration in postmenopausal patients has been 25.

Other tests evaluated in trials include the SET_{ER/PR} index and the SET2,3 indexes. SET2,3 index (commercially developed as Endocrine Activity Index (EAI)) is a 31-gene expression assay that offers prognostic information for patients receiving endocrine therapy. It measures the 28-gene SET_{ER/PR} index of transcription related to estrogen and progesterone receptors, but excluding proliferation-related genes, as well as a baseline prognostic index (BPI) derived from pathologic tumor size, number of involved lymph nodes and molecular subtype by RNA4 (ESR1, PGR, ERBB2, and AURKA) wherein higher BPI represents less aggressive disease. High Endocrine Activity Index (EAI) scores (≥2.10) are associated with endocrine sensitivity and more favorable prognosis, and low Endocrine Activity Index (EAI) scores (<2.10) with endocrine therapy resistance and less favorable outcomes. The assay typically uses a customized multiplex RNA hybridization assay designed for formalin fixed paraffin embedded (FFPE) tumor tissues, e.g., quantagene signal amplification on bDNA, and is highly reproducible results within and between different laboratories. Nonetheless, the test has also been technically validated in qPCR platforms.

The present disclosure provides methods and systems for determining a risk of occurrence or recurrence of breast cancer in a subject. In some aspects, the methods may involve applying a hazard regression analysis to a recurrence score (RS) and an endocrine activity index score (EAI). In some embodiments, the RS may be determined by detected amounts of mRNA expressed from certain genes. These genes may include estrogen receptor 1 (ESR1), progesterone receptor (PGR), BCL2, and SCUBE2 genes. Determining the RS may further involve determining detected amounts of mRNA expressed from additional sets of genes. These additional sets may include a set of proliferation genes comprising one or more of Ki-67, STK15, Survivin, Cyclin B1, and MYBL2 genes, a set of invasion genes comprising one or more of Stromelysis 3 and Cathepsin L2 genes, a set of human epidermal growth factor receptor genes comprising one or more of HER2 and GRB7. The EAI may be determined by detected amounts of mRNA expressed from a plurality of genes. These genes may include SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KCNE4 genes.

Additional details are provided in the following sections.

### I. The Endocrine Activity Index® (EAI®) (Formerly the SET_{ER/PR} Index)

Embodiments of the present disclosure describe a SET_{ER/PR} index. The SET_{ER/PR} index is a measure of the level of transcriptional activity of genes that are related to receptors for the hormones estrogen and progesterone. The SET2,3 index combines the SET index of transcription related to estrogen and progesterone receptors (SET_{ER/PR}) with a baseline prognostic index (BPI) derived from pathologic tumor size, nodal involvement, and molecular subtype by RNA4. A “high” SET2,3 index was shown to be associated with a good prognosis on endocrine therapies.

The SET_{ER/PR} index is calculated using the expression level of a combination of a plurality of genes related to both estrogen receptor (ER) and progesterone receptor (PR), such as disclosed in Table 1 including SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KCNE4. In some aspects, 5, 6, 7, 8, 9, 10, 11, 12, 13, 14, 15, 16, 17, or 18 of the genes in Table 1 are used to determine the SET_{ER/PR} index. The ER- and PR-related genes can be normalized to reference genes, such as disclosed in Table 1 including LDHA, ATP5J2, VDAC2, DARS, UCP2, UBE2Z, AK2, WIPF2, APPBP2, and TRIM2. In some aspects, 1, 2, 3, 4, 5, 6, 7, 8, 9 or 10 of the reference genes disclosed in Table 1 are used to normalize the expression of the ER- and PR-related genes.

TABLE 1
Table 1: ESR1- and PGR-associated genes and reference genes

| Symbol | Name | Entrez ID | Band |
|---|---|---|---|
| SLC39A6 | Solute carrier family 39 (zinc transporter), member 6 | 25800 | 18q12.2 |
| STC2 | Stanniocalcin 2 | 8614 | 5q35.1 |
| CA12 | Carbonic anhydrase XII | 771 | 15q22 |
| ESR1 | Estrogen receptor 1 | 2099 | 6q25.1 |
| PDZK1 | PDZ domain containing 1 | 5174 | 1q21 |
| NPY1R | Neuropeptide Y receptor Y1 | 4886 | 4q32.2 |
| CD2 | CD2 molecule | 914 | 1p13.1 |
| MAPT | Microtubule-associated protein tau | 4137 | 17q21.1 |
| QDPR | Quinoid dihydropteridine reductase | 5860 | 4p15.31 |
| AZGP1 | Alpha-2-glycoprotein-1, zinc-binding | 563 | 7q22.1 |
| ABAT | 4-aminobutyrate aminotransferase | 18 | 16p13.2 |
| ADCY1 | Adenylate cyclase 1 | 107 | 7p12.3 |
| CD3D | CD3D molecule, delta (CD3-TCR complex) | 915 | 11q23 |
| NAT1 | N-acetyltransferase 1 (arylamine N-aminotransferase) | 9 | 8p22 |
| MRPS30 | Mitochondrial ribosomal protein S30 | 10884 | 5q11 |
| DNAJC12 | DNAJ (Hsp40) homolog, subfamily C, member 12 | 56521 | 10q22.1 |
| SCUBE2 | Signal peptide, CUB domain, EGF-like 2 | 57758 | 11p15.3 |
| KCNE4 | Potassium channel, voltage-gated subfamily E regulatory | 23704 | 2q36.1 |
| | subunit 4 | | |
| LDHA | Lactate dehydrogenase A | 3939 | 11p15.4 |
| ATP5J2 | ATP synthase, mitochondrial Fo complex, subunit F2 | 9551 | 7q22.1 |
| VDAC2 | Voltage dependent anion channel 2 | 7417 | 10q22 |
| DARS | Aspartylt tRNA synthetase | 1615 | 2q21.3 |
| UGP2 | UDP-glycose phosphorylase 2 | 7360 | 2p14-p13 |
| UBE2Z | Ubiquitin-conjugating enzyme E2Z | 65264 | 17q21.32 |
| AK2 | Adenylate kinase 2 | 204 | 1p34 |
| WIPF2 | WAS/WASL interacting protein family, member 2 | 147179 | 17q21.2 |
| APPBP2 | Amyloid beta precursor protein (cytoplasmic tail) binding | 10513 | 17q23.2 |
| | protein 2 | | |
| TRIM2 | Tripartite motif containing 2 | 23321 | 4q31.3 |

In some aspects, the SET_{ER/PR} index is calculated as:

$${SET}_{ER / PR} = \frac{{\sum }_{i = 1}^{18} {T}_{i}}{i} - \frac{{\sum }_{j = 1}^{10} {R}_{j}}{j} + 2 ,$$

where Tᵢ is the expression of the ith of the 18 target genes and Rⱼ the expression of the jth of the 10 reference genes. A constant is added to optimize the separation into hormone receptor-positive and negative cases by immunohistochemistry at a score value of 0.

### II. The SET2,3 Index

### Calculation of SET2,3 Index (SET_{ER/PR} Index Adjusted for Baseline Prognostic Index)

Embodiments of the present disclosure describe a SET2,3 index. SET2,3 is calculated as SET_{ER/PR} index adjusted for a baseline prognostic index (BPI) that includes tumor size, nodal involvement, and RNA4. The pT risk score is calculated from largest pathological tumor dimension and assigned score of zero if ≤10 mm, linearly scaled to the range 0-3.0 if measuring 11-39 mm, assigned risk score 3.0 if ≥40 mm, and assigned risk score 3.0 if the tumor otherwise has stage category pT4.

The pN risk score is calculated from the number of involved lymph nodes (0.5 units per node) and assigned score of 5.0 if 10 or more nodes are involved or otherwise stage category pN3. Any lymph node reported as isolated tumor cells (pN0i+) is considered to be negative for this score.

The RNA4 risk score is calculated from the expression of ESR1, ERBB2, PGR, and AURKA. Each transcript is normalized by subtracting the mean of the 10 reference genes and adding the constant of 10. Since ESR1, PGR and ERBB2 have bimodal distributions of gene expression in breast cancers, we defined high ESR1 and PGR expression status as expression exceeding a cut-point two standard deviations (20) below the mean value in the higher expression peak: ESR1=8.93, PGR=5.10. Similarly, we defined the cut-point for ERBB2 gene expression status (11.97) as 2σ above the mean value of gene expression in the lower expression peak. RNA4 risk scores are calculated as the sum of the risk scores from each gene expression measurement.

If PGR-high (PGR>5.10), then calculate AURKA risk score as AURKA expression value minus 7.0, but assign 0 if AURKA<7.0, and assign 2.0 if AURKA>9.0 (range of RNA4 risk score 0-2.0).

If PGR-borderline (4.50<PGR≤5.10), add 1.0 to AURKA risk score (range of RNA4 risk score 1.0-3.0).

If PGR-low (PGR≤4.50), then add 1.0 to AURKA risk score, but AURKA risk score is calculated as AURKA expression value minus 7.5, and assigned 0 if AURKA<7.5, and assigned 1.0 if AURKA>8.5 (range of RNA4 risk score 2.0-3.0).

If ESR1-low (ESR1≤8.93), then add 0.5 to the RNA4 risk score.

If ERBB2-high (ESR1≥11.97), then add 0.5 to the RNA4 risk score.

The baseline prognostic index (BPI) is the sum of risk scores subtracted from 11.0, so: BPI=[11−(pT+pN+RNA4)]×4/11. BPI is zero-truncated (negative values become zero), so BPI has range from 0 to 4.0, and a higher BPI value represents more indolent baseline prognosis.

SET2,3 index is calculated as the weighted sum of SET_{ER/PR} index of hormone receptors-related transcriptional activity and the baseline prognostic index (BPI), as follows: SET2,3=0.75*SET_{ER/PR}+0.51*BPI.

### III. The Recurrence Score (RS)

Embodiments of the present disclosure describe a Recurrence Score index. The Recurrence Score index is calculated using the expression level of a combination of 21 genes related to both estrogen receptor (ER), progesterone receptor (PR), and HER2 such as disclosed in Table 2 and standard histopathologic factors.

TABLE 2

| | | | |
|---|---|---|---|
| | | Entrez | |
| Symbol | Name | ID | Band |

Proliferation

| | | | |
|---|---|---|---|
| Ki-67 | marker of proliferation Ki-67 | 4288 | 10q26.2 |
| STK15 | aurora kinase A | 6790 | 20q13.2 |
| (AURKA) | | | |
| BIRC5 | baculoviral IAP repeat | 332 | 17q25.3 |
| (SURVIVIN) | containing 5 | | |
| CCNB1 | cyclin B1 | 891 | 5q13.2 |
| MYBL2 | MYB proto-oncogene like 2 | 4605 | 20q13.12 |

Invasion

| | | | |
|---|---|---|---|
| MMP11 | Matrix Metallopeptidase 11 | 4320 | 22q11.23 |
| (stromelysin 3) | | | |
| CTSL2 | Cathepsin V | 1515 | 9q22.33 |
| (CTSU) | | | |

HER2 Group

| | | | |
|---|---|---|---|
| HER2 | erb-b2 receptor tyrosine kinase 2 | 2064 | 17q12 |
| (ERBB2) | | | |
| GRB7 | growth factor receptor bound | 2886 | 17q12 |
| | protein 7 | | |

Estrogen

| | | | |
|---|---|---|---|
| ESR1 | Estrogen receptor 1 | 2099 | 6q25.1 |
| PGR | progesterone receptor | 5241 | 11q22.1 |
| BCL2 | BCL2 apoptosis regulator | 596 | 18q21.33 |
| SCUBE2 | Signal peptide, CUB domain, | 57758 | 11p15.3 |
| | EGF-like 2 | | |

Other

| | | | |
|---|---|---|---|
| GSTM1 | glutathione S-transferase mu 1 | 2944 | 1p13.3 |
| CD68 | CD68 molecule | 968 | 17p13.1 |
| BAG1 | BAG cochaperone 1 | 573 | 9p13.3 |
| ACTB | beta actin | 60 | 7p22.1 |
| GAPDH | glyceraldehyde-3-phosphate | 2597 | 12p13.31 |
| | dehydrogenase | | |
| RPLP0 | ribosomal protein lateral stalk | 6175 | 12q24.23 |
| | subunit P0 | | |
| GUS | glucuronidase beta | 2990 | 7q11.21 |
| TFRC | transferrin receptor | 7337 | 3q29 |

In some embodiments, the Recurrence Score incorporates an ER H-score and a PR H-score. ER H-score and a PR H-score are methods used to evaluate the expression of estrogen receptors (ER) and progesterone receptors (PR) in breast cancer tissues. These scores help determine the likelihood of a patient's response to hormone therapy, typically by determining the staining intensity of tumor cells. In determining the ER H-score and the PR H-score tumor cells are stained and categorized based on the intensity of the staining according to the following scale: 0: No staining; 1: Weak staining; 2: Moderate staining and 3: Strong staining. The percentage of cells at each staining intensity is determined by multiplying the percentage of cells by the corresponding intensity score and then summing these values. The formula is:

$$H \text{-score} = ( \% of cells with intensity 1 \times 1 ) + ( \% of cells with intensity 2 \times 2 ) + ( \% of cells with intensity 3 \times 3 ) .$$

The maximum possible H-score is 300, and an H-score of 100 or more typically indicates a positive result, suggesting that the patient may benefit from hormone therapy.

In some embodiments, the Recurrence Score incorporates other histology scores. The Nottingham histologic score (or histologic grade) provides a scoring system to assess the “grade” of breast cancers. The grade is a way to rate how aggressive a tumor may behave. Typically, Nottingham provides a total of 3 different scores. The pathologist looks at the breast cancer cells under a microscope and gives a score to 3 characteristics:
Tubule formation—or how much the tumor looks like normal cell structure.
Nuclear pleomorphism—how different the tumor cells look from normal cells.
Mitotic activity—or how fast cells are dividing or reproducing.

Each characteristic is given a score from 1 to 3, with 1 being the closest to normal and 3 being the most abnormal. These 3 scores are added together, making the Nottingham Score. The minimum score possible is 3 (1+1+1) and the maximum possible is 9 (3+3+3). The total score is then assigned to a grade:
Grade I is assigned for a total score of 3 to 5. This is also called well differentiated.
Grade II is assigned for a total score of 6 to 7. This is also called moderately differentiated.

Grade III is assigned for a total score of 8-9. This is also called poorly differentiated. Grade I cancers tend to be less aggressive. They are also more often estrogen receptor-positive (ER+). Grade III cancers tend to be more aggressive and are more likely to be “triple-negative”− or negative for hormone (ER & PR) and HER2 receptors.

Magee equations (MEs) are a set of multivariable models that were developed to estimate the actual Oncotype DX (ODX) recurrence score in invasive breast cancer. The equations were derived from standard histopathologic factors and semiquantitative immunohistochemical scores of routinely used biomarkers. The 3 equations use slightly different parameters but provide similar results. ME1 uses Nottingham score, tumor size, and semiquantitative results for estrogen receptor (ER), progesterone receptor, HER2, and Ki-67. ME2 is similar to ME1 but does not require Ki-67. ME3 includes only semiquantitative immunohistochemical expression levels for ER, progesterone receptor, HER2, and Ki-67. Several studies have validated the clinical usefulness of MEs in routine clinical practice.

Both single and multi-institutional studies have shown that the rate of pathologic complete response (pCR) to neoadjuvant chemotherapy in ER+/HER2-negative patients can be predicted by ME3 scores. The estimated pCR rates are 0%, <5%, 14%, and 35 to 40% for ME3 score<18, 18 to 25, >25 to <31, and 31 or higher, respectively.

The original Magee equation is represented as follows: recurrence score=13.424+5.420*(nuclear grade)+5.538*(mitotic count)−0.045*(ER H-score)−0.030*(PR H-score)+9.486*(0 for negative/equivocal and 1 for HER2 positive) on a set of 42 cases. ER H-score is a semi quantitative measure of the intensity and distribution of estrogen receptor (ER) staining in breast cancer tissue. It is calculated by adding the percentage of ER-positive tumor cells at each intensity level multiplied by an ordinal value for that intensity level. Similarly, PR-H is a semi quantitative measure of the intensity and distribution of progesterone receptor (PR) staining in breast cancer tissue. Scores range from 0 to 300 with higher scores indicating stronger staining.

Subsequently, a new equation was built based on over 800 cases with histopathologic data and Recurrence Scores between 2004 and 2009. The three new equations are as follows:

$$New Magee equation 1 :$$

$$Recurrence Score = 15.31385 + Nottingham Score * 1.4055 + ERIHC * ( - 0.01924 ) + PRIHC * ( - 0.02925 ) + ( 0 for HER 2 negative , 0.77681 for equivocal , 11.58134 for HER 2 positive ) + tumor size * 0.78677 + Ki - 67 index * 0.13269 .$$

$$New Magee equation 2 :$$

$$Recurrence Score = 18.8042 + Nottingham Score * 2.314123 + ERIHC * ( - 0.03749 ) + PRIHC * ( - 0.03065 ) + ( 0 for HER 2 negative , 1.82921 for equivocal , 11.51378 for HER 2 positive ) + tumor size * 0.04267 .$$

$$New Magee equation 3 :$$

$$Recurrence Score = 24.30812 + ERIHC * ( - 0.02177 ) + PRIHC * ( - 0.02884 ) + ( 0 for HER 2 negative , 1.46495 for equivocal , 12.75525 for HER 2 positive ) + Ki - 67 * 0.18649 .$$

### IV. Sample Analysis

a. Isolation of RNA

Aspects of the present disclosure concern the isolation of RNA from a patient sample for use in determining the SET_{ER/PR} index, the SET2,3 index, and the Recurrence Score (RS). The patient sample may blood, saliva, urine, or a tissue biopsy. The tissue biopsy may be a tumor biopsy that has been flash-frozen (e.g. in liquid nitrogen), formalin-fixed and paraffin-embedded (FFPE), and/or preserved by a RNA stabilization agent (e.g., RNAlater). In some aspects, isolation is not necessary, and the assay directly utilizes RNA from within a homogenate of the tissue sample. In certain aspects the homogenate of FFPE tumor sample is enzymatically digested.

RNA may be isolated using techniques well known to those of skill in the art. Methods generally involve lysing the cells with a chaotropic (e.g., guanidinium isothiocyanate) and/or detergent (e.g., N-lauroyl sarcosine) prior to implementing processes for isolating particular populations of RNA. Chromatography is a process often used to separate or isolate nucleic acids from protein or from other nucleic acids. Such methods can involve electrophoresis with a gel matrix, filter columns, coated magnetic beads, alcohol precipitation, and/or other chromatography.

b. Gene Expression Assessment

In certain aspects, methods of the present disclosure concern measuring expression of ER- and PR-related genes as well as one or more reference genes in a sample from a subject with breast cancer. The expression information may be obtained by testing cancer samples by a lab, a technician, a device, or a clinician. In a certain embodiment, the differential expression of one or more genes described herein may be measured.

Expression levels of the genes can be detected using any suitable means known in the art. For example, detection of gene expression can be accomplished by detecting nucleic acid molecules (such as RNA) using nucleic acid amplification methods (such as RT-PCR, droplet-based RT amplification, exon capture of RNA sequence library, next generation RNA sequencing), array analysis (such as microarray analysis), or hybridization methods (such as ribonuclease protection assay, bead-based assays, or Nanostring®. Detection of gene expression can also be accomplished using assays that detect the proteins encoded by the genes, including immunoassays (such as ELISA, Western blot, RIA assay, or protein arrays).

The pattern or signature of expression in each cancer sample may then be used to generate a cancer prognosis or classification, such as predicting cancer survival or recurrence, using the SET_{ER/PR} index. The expression of one or more of ER- and PR-related genes could be assessed to predict or report prognosis or prescribe treatment options for cancer patients, especially breast cancer patients.

The expression of one or more ER- and PR-related genes may be measured by a variety of techniques that are well known in the art. Quantifying the levels of the messenger RNA (mRNA) of a gene may be used to measure the expression of the gene. Alternatively, quantifying the levels of the protein product of ER- and PR-related genes may be to measure the expression of the genes. Additional information regarding the methods discussed below may be found in Ausubel et al., (2003) Current Protocols in Molecular Biology, John Wiley & amp; Sons, New York, NY, or Sambrook et al. (1989) Molecular Cloning: A Laboratory Manual, Cold Spring Harbor Press, Cold Spring Harbor, NY. One skilled in the art will know which parameters may be manipulated to optimize detection of the mRNA or protein of interest.

The steps of a representative protocol for quantitating gene expression level using fixed, paraffin-embedded tissues as the RNA source, including mRNA isolation, purification, primer extension and amplification are given in various published journal articles (see Godfrey et al., 2000; Specht et al., 2001). Briefly, a representative process starts with cutting about 10 μm thick sections of paraffin-embedded neoplasm tissue samples or adjacent non-cancerous tissue. The RNA is then extracted, and protein and DNA are removed. Alternatively, RNA is isolated directly from a neoplasm sample or other tissue sample. After analysis of the RNA concentration, RNA repair and/or amplification steps can be included, if necessary, and RNA is reverse transcribed using gene specific primers, followed by preparation of a tagged RNA sequencing library, and paired-end sequencing. In another example, the RNA is not reverse transcribed, but is directly hybridized to a specific template and then labeled with oligonucleotides and/or chemical or fluorescent color to be detected and counted by a laser.

Immunohistochemical staining may also be used to measure the differential expression of a plurality of ER- and PR-related genes. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

A nucleic acid microarray may be used to quantify the differential expression of a plurality of ER- and PR-related genes. Microarray analysis may be performed using commercially available equipment, following manufacturer's protocols, such as by using the Affymetrix GeneChip® technology (Santa Clara, CA) or the Microarray System from Incyte (Fremont, CA), or a QuantiGene® Signal amplification method on bDNA. Typically, single-stranded nucleic acids (e.g., cDNAs or oligonucleotides) are plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with a robust statistical normalization algorithm to generate expression values.

Quantitative real-time PCR (qRT-PCR) may also be used to measure the differential expression of a plurality of ER- and PR-related genes. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

For example, extracted RNA can be reverse-transcribed using a GeneAmp® RNA PCR kit (Perkin Elmer, Calif., USA), following the manufacturer's instructions. In some embodiments, gene expression levels can be determined using a gene expression analysis technology that measure mRNA in solution. Methods of detecting gene expression are described for example in U.S. Patent Application Nos. US20140357660, and US20130259858; incorporated herein by reference. Examples of such gene expression analysis technologies include, but not limited to RNAscope™, RT-PCR, Nanostring®, QuantiGene®, gNPA®, HTG®, microarray, and sequencing. For example, methods of Nanostring use labeled reporter molecules, referred to as labeled “nanoreporters,” that are capable of binding individual target molecules. Through the nanoreporters label codes, the binding of the nanoreporters to target molecules results in the identification of the target molecules. Methods of Nanostring are described in U.S. Pat. No. 7,473,767 (see also, Geiss et al., 2008). Methods may include the RainDance droplet amplification method such as described in U.S. Pat. No. 8,535,889, incorporated herein by reference. Sequencing may include exon capture, such as Illumina targeted sequencing after the generation of a tagged library for next generation sequencing (e.g. described in International Patent Application No. WO2013131962, incorporated herein by reference).

A non-limiting example of a fluorescent reporter probe is a TaqMan® probe (Applied Biosystems, Foster City, CA). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR is typically performed using a reference standard. The ideal reference standard is expressed at a constant level among different tissues, and is unaffected by the experimental treatment. The system can include a thermocycler, laser, charge-coupled device (CCD) camera, and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by an experimental treatment. RNAs commonly used to normalize patterns of gene expression are mRNAs for the housekeeping genes GAPDH, β-actin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems (Foster City, CA).

An enzyme-linked immunosorbent assay, or ELISA, may be used to measure the differential expression of a plurality of ER- and PR-related genes. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of ER- and PR-related genes. For this, a plurality of antibodies is arrayed and covalently attached to the surface of the microarray or biochip. A protein extract containing the biomarker proteins of interest is generally labeled with a fluorescent dye.

The labeled ER- and PR-related genes proteins may be incubated with the antibody microarray. After washes to remove the unbound proteins, the microarray is scanned. The raw fluorescent intensity data may be converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up 100 may be detected in one well. The small size/surface area of the beads and the three dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope.

Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

### V. Integration of Endocrine Activity Index and Recurrence Scores

In many aspects, the disclosure provides methods and processes for determining a risk of recurrence or occurrence of breast cancer in a subject, the methods comprising applying a hazard regression analysis to detected amounts of a set of biomarkers. The biomarkers can be detected with a variety of methods, e.g., in some instances the biomarkers can be detected via probe hybridization, in other instances the biomarkers can be detected with semiquantitative immunohistochemical expression levels of, e.g., sets of estrogen receptor (ER) genes, e.g., sets of progesterone receptor genes, e.g., HER2, and e.g., Ki-67 proliferation markers. In some aspects, the disclosure relates to processes and methodology employed in developing hazard regression models applied in the comparative evaluation of various approaches for assessing a risk of occurrence or recurrence of breast cancer. The disclosure further relates to the discovery of novel processes and associated metrics for determining distant recurrence-free interval (DRFI) and overall survival (OS) in patients that received endocrine therapy (ET). The disclosure provides a framework for determining a risk of breast cancer in a subject, and demonstrates that an integration of inputs from different sets of markers provides a significantly larger contribution to prediction of risk of a distant recurrence (prognosis) and overall survival.

In some embodiments that can be combined with the previous embodiments, the disclosure provides a method for determining a risk of recurrence of breast cancer in a subject, the method comprising: applying a hazard regression analysis to: a recurrence score (RS) determined by a detected amount of a set of mRNAs expressed from a set of genes comprising the estrogen receptor 1 (ESR1), the progesterone receptor (PGR), BCL2, and SCUBE2 genes, whereby the mRNA is extracted from a breast tissue sample; and an endocrine activity index score (EAI) determined by a detected amount of a plurality of genes in a set of mRNAs comprising at least 5 genes, at least 6 genes, at least 7 genes, at least 8 genes, at least 9 genes, at least 10 genes, at least 11 genes, at least 12 genes, at least 13 genes, at least 14 genes, at least 15 genes, at least 16 genes, at least 17 genes, at least 18 genes in a set of mRNAs comprising SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KCNE4 genes; whereby the mRNA is extracted from a breast tissue sample.

In some embodiments that can be combined with the previous embodiments, the disclosure provides a method for determining a risk of recurrence of breast cancer in a subject, the method comprising: determining the mRNA expression levels of a panel of biomarkers comprising at least 19 biomarkers, at least 20 biomarkers, at least 21 biomarkers, at least 22 biomarkers, at least 23 biomarkers, at least 24 biomarkers, at least 25 biomarkers, at least 26 biomarkers, at least 27 biomarkers, at least 28 biomarkers, at least 29 biomarkers, or 30 biomarkers selected from selected from the estrogen receptor 1 (ESR1), the progesterone receptor (PGR), BCL2, Ki-67, STK₁₅, Survivin, Cyclin B₁, MYBL₂, Stromelysis 3 Cathepsin L₂, HER₂, GRB₇, SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KCNE4 genes; and identifying a risk of breast cancer in the subject by applying a multivariate proportional hazards regression model to the outputs of the mRNA expression levels of the panel of biomarkers.

In some embodiments that can be combined with the previous embodiments the set of genes informing the recurrence score (RS) and/or the endocrine activity index (EAI) may further comprise a set of proliferation genes comprising one or more of Ki-67, STK₁₅, Survivin, Cyclin B₁, and MYBL₂ genes, a set of invasion genes comprising one or more of Stromelysis 3 and Cathepsin L₂ genes, a set of human epidermal growth factor receptor genes comprising one or more of HER₂ and GRB₇. In some embodiments that can be combined with the previous embodiments the set of genes informing the recurrence score (RS) and/or the endocrine activity index (EAI) may be normalize to one or more reference genes, e.g., Beta-actin, GAPDH, RPLPO, GUS, TFRC, MAPT, CD2, CD3D, QDPR, PDZK1, STC2, MRPS30, DNAJC12, SCUBE2, and KCNE4 genes. A variety of genes can be used as reference genes, and one or all genes can be selected from the set of reference genes. In some aspects, the set of genes determining the endocrine activity index score is a set of ten genes from the group consisting of SLC39A6, STC2, CA12, ESR1, PDZK1, NPY1R, CD2, MAPT, QDPR, and AZGP1 genes, a set of ten genes from the group consisting of QDPR, AZGP1, ABAT, ADCY1, CD3D, NAT1, MRPS30, DNAJC12, SCUBE2, and KCNE4 genes.

Continuing with the formula for determining the EAI, it may be represented as follows: Σ(i=1 to n) Ti−Σ(j=1 to m) Rj+C, where ‘n’ represents the total number of target genes, ‘m’ represents the number of reference genes, ‘Ti’ represents the detected mRNA levels of each target gene, ‘Rj’ represents the detected mRNA levels of each reference gene, and ‘C’ is a constant that may be adjusted based on empirical data. When a total of 18 genes and 10 informative genes are selected, the endocrine activity index score (EAI) is determined by

$$EAI = \frac{{\sum }_{i = 1}^{18} Ti}{i} - \frac{{\sum }_{j = 1}^{10} Rj}{j} + 2.$$

It will be understood that when a lesser cohort of genes is selected, the sums in the formula can be accordingly adjusted, e.g. 17 biomarkers

$$EAI = \frac{{\sum }_{i = 1}^{17} Ti}{i} - \frac{{\sum }_{j = 1}^{10} Rj}{j} + 2 ,$$

16 biomarkers

$$EAI = \frac{{\sum }_{i = 1}^{16} Ti}{i} - \frac{{\sum }_{j = 1}^{10} Rj}{j} + 2 ,$$

15 biomarkers

$$EAI = \frac{{\sum }_{i = 1}^{15} Ti}{i} - \frac{{\sum }_{j = 1}^{10} Rj}{j} + 2 ;$$

14 biomarkers

$$EAI = \frac{{\sum }_{i = 1}^{14} Ti}{i} - \frac{{\sum }_{j = 1}^{10} Rj}{j} + 2 ,$$

13 biomarkers

$$EAI = \frac{{\sum }_{i = 1}^{13} Ti}{i} - \frac{{\sum }_{j = 1}^{10} Rj}{j} + 2 ;$$

12 biomarkers

$$EAI = \frac{{\sum }_{i = 1}^{12} Ti}{i} - \frac{{\sum }_{j = 1}^{10} Rj}{j} + 2 ,$$

11 biomarkers

$$EAI = \frac{{\sum }_{i = 1}^{11} Ti}{i} - \frac{{\sum }_{j = 1}^{10} Rj}{j} + 2 ,$$

or 10 biomarkers

$$EAI = \frac{{\sum }_{i = 1}^{10} Ti}{i} - \frac{{\sum }_{j = 1}^{10} Rj}{j} + 2 .$$

In some cases, the recurrence score is determined by a detected amount of mRNA expressed from a set of genes, e.g., the ESR1, PGR, BCL2 gene, SCUBE2, and/or other RS genes inputted into the formula: 13.424+5.420*(nuclear grade)+5.538*(mitotic count)−0.045*(ER H-score)−0.030*(PR H-score)+9.486*(0 for negative/equivocal and 1 for HER2 positive). Similarly, a RS can also be determined with another set of Magee Equations. In some cases, hazard regression model is a Cox proportional hazard model for distant recurrence-free interval (DRFI).

In some embodiments that can be combined with the previous embodiments, the disclosure provides a method for determining a risk of recurrence of breast cancer in a subject, the method comprising: applying a Cox proportional hazard regression analysis to a set of Set ER/PR, Set2,3, and or Recurrence Score (RS) parameters. In some instances, the Cox proportional hazard model is

$$h ( t ) ≅ {h}_{0} ( t ) exp ( {β}_{1} R S + {β}_{2} EAI Index + {β}_{3} BPI ) \qquad EQ . 1$$

where β₁, β₂, and β₃, are the (natural) logarithms of the hazard rates for RS, EAI and BPI, respectively, and h₀(t) is the baseline hazard function. In some embodiments the distant recurrence-free interval (DRFI) is determined by:

$${Linear}_{DRFI} = 0.0 \; 113 RS - 0 \text{.54} EAI - 0 \text{.80} BPI \qquad EQ . 2$$

In some embodiments that can be combined with previous embodiments, the distant recurrence-free interval (DRFI) is provided on a scale from 0 to 100 determined by:

$${Compressed}_{DRFI} = \{ \begin{matrix}- 4.8 & if {Linear}_{DRFI} < - 4.8 \\ 1.1 & if {Linear}_{DRFI} > 1.1 \\ {Linear}_{DRFI} & otherwise\end{matrix} \qquad EQ . 4$$

$${SET}_{DRFI} = \frac{100}{( 1.10 + 4.80 )} ( {Compressed}_{DRFI} + 4.80 ) \qquad EQ . 6$$

In some embodiments that can be combined with previous embodiments, an overall survival (OS) of the subject is determined by:

$${Linear}_{OS} = 0.0 \; 104 RS - 0 \text{.71} EAI - 0 \text{.80} BPI \qquad EQ . 3$$

In some embodiments that can be combined with previous embodiments, the overall survival (OS) is provided on a scale from 0 to 100 determined by:

$${Compressed}_{OS} = \{ \begin{matrix}- 5.3 & if {Linear}_{OS} < - 5.3 \\ 1.1 & if {Linear}_{OS} > 1.1 \\ {Linear}_{OS} & otherwise\end{matrix} \qquad EQ . 5$$

$${SET}_{O S} = \frac{100}{( 1.10 + 5.30 )} ( {Compressed}_{OS} + 5.30 ) \qquad EQ . 7$$

In some embodiments that can be combined with previous embodiments, the distant recurrence-free interval (DRFI) and/or the overall survival (OS) of the subject is determined by:

$${Linear}_{DRFI} = 0.0113 RS - 0 \text{.54} {SET}_{ER / PR} Index \qquad EQ . 9$$

$${Linear}_{OS} = 0.0 \; 104 RS - 0 \text{.71} {SET}_{ER / PR} Index \qquad EQ . 10$$

In some embodiments that can be combined with previous embodiments, the compressed distant recurrence-free interval (DRFI) and/or the overall survival (OS) of the subject is determined by:

$${Compressed}_{DRFI} = \{ \begin{matrix}- 1.62 & if {Linear}_{DRFI} < - 1.62 \\ 1.13 & if {Linear}_{DRFI} > 1.13 \\ {Linear}_{DRFI} & otherwise\end{matrix} \qquad EQ . 11$$

$${Compressed}_{OS} = \{ \begin{matrix}- 5.3 & if {Linear}_{OS} < - 2.13 \\ 1.04 & if {Linear}_{OS} > 1.04 \\ {Linear}_{OS} & otherwise\end{matrix} \qquad EQ . 12$$

In some embodiments that can be combined with previous embodiments, the compressed distant recurrence-free interval (DRFI) and/or the overall survival (OS) of the subject is provided on a scale from 0 to 100 determined by:

$${SET}_{DRFI} = \frac{100}{( 1.13 + 1.62 )} ( {Compressed}_{DRFI} + 1.62 ) \qquad EQ . 6$$

$${SET}_{OS} = \frac{100}{( 1.04 + 2.13 )} ( {Compressed}_{OS} + 2.13 ) \qquad EQ . 7$$

In some embodiments that can be combined with previous embodiments, the mRNA expression levels are determined with a set of probe pairs each modified with a detectable moiety. The detectable moiety can be, e.g., a biotin moiety or a fluorescent moiety. In some embodiments, the mRNA expression levels are determined via hybridization of one or more probes complementary to the target mRNA genes in a branched DNA (bDNA) assay. The bDNA assay can comprise sequential hybridization of the bDNA pre-amplifier, amplifier, and probe molecules labeled with a detectable moiety to the target mRNA.

In some embodiments that can be combined with previous embodiments, the hazard regression analysis provides a patient specific score for the subject. In some embodiments that can be combined with previous embodiments, the hazard regression analysis provides a population specific score for the subject.

### VI. Reagents and Kits

Further embodiments of the invention include kits for the measurement, analysis, and reporting of gene expression and transcriptional output of the sets of genes described herein. The present disclosure also provides reaction mixtures comprising reagents and methods for detecting the sets of genes described herein. A kit may include, but is not limited to probe sets, microarray, quantitative RT-PCR, or other genomic platform reagents and materials, as well as hardware and/or software for performing at least a portion of the methods described. For example, custom modified probes (e.g., bDNA probe sets or primer pairs for targeted amplification of one or more biomarker) are contemplated. Accordingly, an article of manufacture or a kit is provided comprising a customized set of modified probes for detecting a plurality of biomarkers. The article of manufacture or kit can further comprise a package insert comprising instructions for using the customized assay to determine, e.g., an integrated input of the SET_{ER/PR} index with a Recurrence Score, an integrated input of the SET2,3 index with a Recurrence Score, or an integrated input of a score that is created by combining a plurality of genes described here. The integrated score can then be used to treat or delay progression of breast cancer in an individual. Probes for any of the biomarkers described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use, including the instructions for integrating scores. The article of manufacture or kit may further include other materials desirable from a commercial and user standpoint, including other buffers, diluents, filters, needles, syringes, and package inserts with instructions for use. Suitable containers for the one or more agent include, for example, bottles, vials, bags and syringes.

In some embodiments, the disclosure provides a reaction mixture comprising: a set of nucleic acid probes, each modified with at least one detectable moiety, for determining the mRNA expression levels of a panel of biomarkers comprising at least 19 biomarkers selected from Ki-67, STK15, Survivin, CCBB1 (cyclin B1), MYBL₂, SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KONE from a biological sample. In some embodiments, the biological sample is obtained from a human, e.g., a breast sample. In some embodiments the reaction mixture further comprises a set of nucleic acid probes, each modified with at least one detectable moiety, for determining the mRNA expression levels of a set of invasion genes comprising one or more of Stromelysis 3 and Cathepsin L₂ genes. In some embodiments the reaction mixture further comprises a set of nucleic acid probes, each modified with at least one detectable moiety, for determining the mRNA expression levels of a set of human epidermal growth factor receptor genes comprising one or more of HER₂ and GRB₇. In some embodiments the reaction mixture further comprises a set of nucleic acid probes, each modified with at least one detectable moiety, for determining the mRNA expression levels of a set of reference genes comprising one or more of Beta-actin, GAPDH, RPLPO, GUS, TFRC, MAPT, CD2, CD3D, QDPR, PDZK1, STC2, MRPS30, DNAJC12, SCUBE2, and KCNE4 genes.

In some embodiments, the disclosure provides a method of sample analysis comprising: subjecting a reaction mixture comprising a set of nucleic acid probes, each modified with at least one detectable moiety, for determining the mRNA expression levels of a panel of biomarkers comprising at least 19 biomarkers selected from Ki-67, STK15, Survivin, CCBB1 (cyclin B1), MYBL2, SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KONE to conditions that allow for hybridization of the set of nucleic acid probes to a set of target nucleic acids from a biological sample or to conditions that allow for amplification of the set of nucleic acid probes to the set of target nucleic acids from; and detecting a signal from the detectable moiety.

These kits may comprise a set of nucleic acid probes, each modified with at least one detectable moiety, for determining the mRNA expression levels of a panel of biomarkers. The panel of biomarkers may comprise at least 19 biomarkers selected from Ki-67, STK15, Survivin, CCBB1 (cyclin B1), MYBL₂, SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KCNE.

In some embodiments, each probe in the set of nucleotide probes may comprise at least 15 contiguous nucleotides from the 3′ end of a nucleic acid sequence as set forth in Tables 1 and Table 2, or their complements. The oligonucleotides may be in one or more containers. When the probes are used to amplify a target gene in an amplification reaction, e.g., qPCR, they typically comprise forward and reverse primers. At least one such primer can be modified with a targeting moiety, but typically both primers are modified with a targeting moiety to amplify the signal. In instances where the detection assay is a hybridization reaction, the probe may not require a forward and a reverse primer.

In some aspects, each probe in the set of nucleotide probes may be in a separate container. In other aspects, each probe in the set of nucleotide probes may be in the same container. In some embodiments, the kit may further comprise a thermostable polymerase. In other embodiments, the kit may further comprise deoxynucleotide triphosphates.

While certain embodiments have been described, these embodiments have been presented by way of example only and are not intended to limit the scope of the present disclosures. Indeed, the novel methods, integration calculations, approaches to data integration, and systems described herein can be embodied in a variety of other forms; furthermore, various omissions, substitutions and changes in the form of the methods, novel methods, integration calculations, approaches to data integration, and systems described herein can be made without departing from the spirit of the present disclosures. The accompanying claims and their equivalents are intended to cover such forms or modifications as would fall within the scope and spirit of the present disclosures.

### EXAMPLES

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods a within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

### Example 1: Combination of Predicted Sensitivity to Endocrine Therapy (SET2,3 Index) and the Recurrence Score (RS) in Node-Positive Breast Cancer: Independent Validation in the PACS-01 Trial

This example details the development of new methodology for determining independent recurrence-free interval (DRFI) and overall survival (OS) from the SET2,3 index and Recurrence Score (RS) indexes.

### Assays:

SET2,3 index measures hormone receptor-related transcriptional activity (SET_{ER/PR}; an 18-gene assay typically normalized to 10 reference genes) adjusted for a baseline prognosis index (BPI) derived from tumor size, nodes involved and a 4-gene molecular subtype (RNA4). The Recurrence Score (RS) is a 21-gene assay that generates a Recurrence Score for each tumor sample, based on expression levels of 16 breast cancer-related genes, normalized to 5 reference genes. The Recurrence Score is a continuous variable that provides an individualized estimate of 9-year distant recurrence risk and the likelihood of adjuvant chemotherapy benefit. Oncotype DX Breast Recurrence Score® incorporated the recurrence score and has been extensively validated Mol. Diagn. Ther. 2020 October; 24 (5): 621-632. Recently, it has been suggested that the SET2,3 could add new information to the 21-gene Breast Recurrence Score (RS) in the SWOG 8814 trial (J Clin Oncol. 2023 Apr. 1; 41 (10): 1841-1848).

The instant examples describe the process development for methods and models revealing novel processes informing distant recurrence-free interval (DRFI) and overall survival (OS) performance from a combination of Recurrence Score (RS) and Set2,3 metrics.

RS and SET2,3 Data from PACS-01 Trial:

Between June 1997 and March 2000, 1,999 patients with operable node-positive breast cancer were randomly assigned to either FEC every 21 days for six cycles, or three cycles of FEC followed by three cycles of docetaxel, both given every 21 days. Hormone-receptor-positive patients received tamoxifen for 5 years after chemotherapy. The primary end point of the trial was 5-year disease-free survival (DFS).

Of 791 tumor samples, 760 (96.1%) passed quality control (QC) for the SET assay; 724 had pathologic information to calculate SET2,3 index; and 659 had results for both RS and SET2,3. Thus, results from 659 samples were available for evaluation of processes considering the performance of both tests, alone and in combination. At a median follow-up of 8 years, distant recurrence occurred in 144 of 659 (21.9%) patients overall, and 94 of 490 (19.2%) patients in the sensitivity population.

### Hazard Regression Analysis

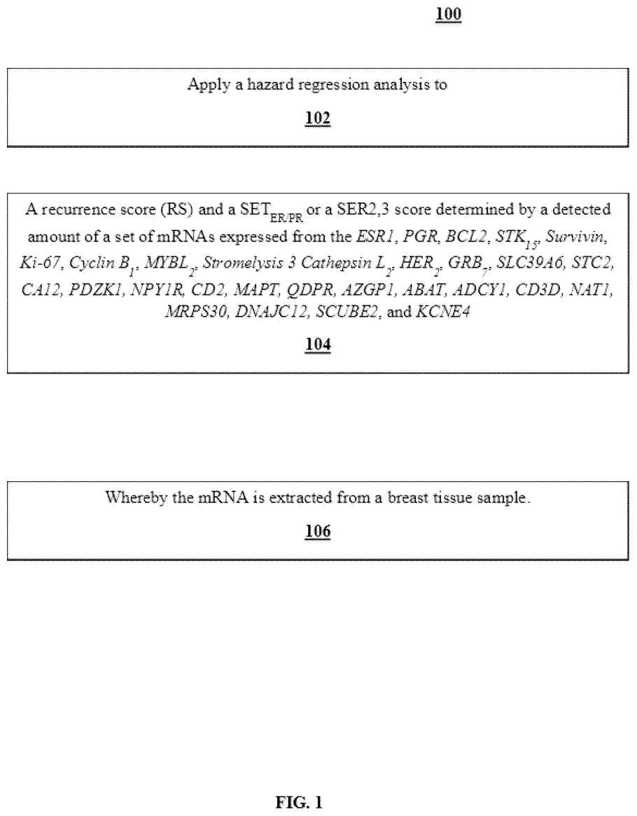

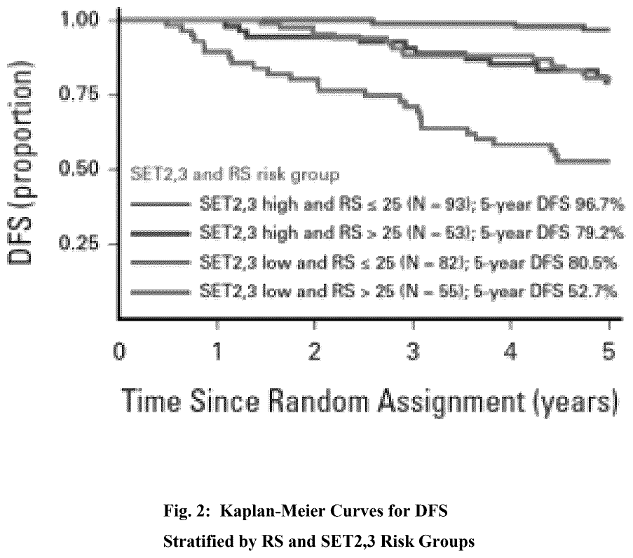

The present study applied a Cox Proportional Hazards Regression Analysis to various datapoints from the PACS-01 659 samples available results for both RS and SET2,3. See FIGS. 1 and 2. The process development considered models for improved prediction of risk of distance recurrence and overall survival using combined information from OncotypeDX Recurrence Score (RS) and the SET2,3 test), as described below. The process development also considered individual performance of each test and seek to identify the most informative parameters on a patient specific level and on a population specific level.

### Linear Predictors of DRFI and OS Based on SET_{ER/PR} Index and BPI

Table 3 provides results of multivariate Cox proportional hazards regression for distant recurrence-free interval (DRFI) and overall survival (OS) in patients that received endocrine therapy (ET). Results suggest that SET_{ER/PR} Index and Baseline Prognostic Index (BPI), collectively, the Set2,3 index, are significantly associated with DRFI after adjustment for RS and treatment.

TABLE 3
Table 3: Multivariate Model of Individual Signatures in
the Sensitivity Analysis of Patients who Received ET.

| Multivariable Cox Model | | DRFI | | | Overall Survival | | |
|---|---|---|---|---|---|---|---|
| Variables | Increment | HR | 95% CI | p-value | HR | 95% CI | p-value |
| Recurrence | per 10 | 1.12 | 0.99- | 0.080 | 1.11 | 0.96- | 0.156 |
| Score | units | | 1.28 | | | 1.29 | |
| SET_{ER/PR} | per unit | 0.58 | 0.36- | 0.019 | 0.49 | 0.28- | 0.009 |
| Index | | | 0.91 | | | 0.83 | |
| Baseline | per unit | 0.45 | 0.34- | <0.001 | 0.45 | 0.33- | <0.001 |
| Prognostic | | | 0.58 | | | 0.61 | |
| Index | | | | | | | |
| Treatment | vs. FEC | 1.19 | 0.79- | 0.398 | 0.96 | 0.60- | 0.862 |
| Arm | (ref) | | 1.80 | | | 1.54 | |

Consider the baseline hazard function h₀(t), which is a function of time, t. The baseline hazard represents that baseline hazard rate when all the predictor variables in the Cox proportional hazards regression model are set to zero (0). Given that the treatment effect is non-significant, the Cox proportional hazards model may be approximated as

$$h ( t ) ≅ {h}_{0} ( t ) exp ( {β}_{1} RS + {β}_{2} {SET}_{ER / PR} Index + {β}_{3} BPI ) \qquad EQ . 1$$

where β₁, β₂, and β₃, are the (natural) logarithms of the hazard rates for RS, SET_{ER/PR} Index and BPI, respectively, and h₀(t) is the baseline hazard function. The approximate coefficients and standard errors for model terms are provided in Table 4.

TABLE 4
Table 2: Coefficients and Standard Errors for Linear Predictor

| | DRFI | | | | Overall Survival | | | |
|---|---|---|---|---|---|---|---|---|
| | | Standard | | | | Standard | | |
| Variables | Coefficient | Error | Z | p-value | Coefficient | Error | Z | p-value |
| Recurrence | 0.0113 | 0.0065 | 1.7505 | 0.080 | 0.0104 | 0.0073 | 1.4187 | 0.156 |
| Score | | | | | | | | |
| SET_{ER/PR} | −0.5400 | 0.2304 | −2.3427 | 0.019 | −0.7100 | 0.2712 | −2.618 | 0.009 |
| Index | | | | | | | | |
| Baseline | −0.8000 | 0.1363 | −5.8694 | <0.001 | −0.8000 | 0.1363 | −5.8694 | <0.001 |
| Prognostic | | | | | | | | |
| Index | | | | | | | | |

Consequently, linear predictors of DRFI and OS can be developed as follows:

$${Linear}_{DRFI} = 0.0113 RS - 0 \text{.54} {SET}_{ER / PR} Index - 0.8 BPI \qquad EQ . 2$$

$${Linear}_{OS} = 0.0 \; 104 RS - 0 \text{.71} {SET}_{ER / PR} Index - 0.8 BPI \qquad EQ . 3$$

Assuming independence of RS, SET_{ER/PR}, and BPI the approximate standard errors per unit of the linear predictors Linear_{DRF1} and Linear_{OS} are approximately 0.1654 and 0.4599, respectively. The ranges of the RS, SET_{ER/PR} Index, and BPI in the PACS-01 study are 0 to 100, 0 to 3, and 0 to 4, respectively. Consequently, Linear_{DRF} and Linear_{OS} range from −4.82 to 1.12 and from −5.30 to 1.11, respectively. To prevent outliers, the tails of the linear predictors are compressed as follows:

$${Compressed}_{DRFI} = \{ \begin{matrix}- 4.82 & if {Linear}_{DRFI} < - 4.82 \\ 1.13 & if {Linear}_{DRFI} > 1.13 \\ {Linear}_{DRFI} & otherwise\end{matrix} \qquad EQ . 4$$

$${Compressed}_{OS} = \{ \begin{matrix}- 5.3 & if {Linear}_{OS} < - 5.33 \\ 1.04 & if {Linear}_{OS} > 1.04 \\ {Linear}_{OS} & otherwise\end{matrix} \qquad EQ . 5$$

The predictors of DRFI and OS are subsequently rescaled from 0 to 100 as follows:

$${SET}_{DRFI} = \frac{100}{( 1.13 + 4.82 )} ( {Compressed}_{DRFI} + 4.82 ) \qquad EQ . 6$$

$${SET}_{OS} = \frac{100}{( 1.04 + 5.33 )} ( {Compressed}_{OS} + 5.33 ) \qquad EQ . 7$$

The analyses above is presented in terms of SET2,3, which can be determined as a linear combination of SET_{ER/P} and BPI; namely,

$$SET 2 , 3 = 0.75 {SET}_{ER / PR} Index + 0.5 BPI \qquad EQ . 8$$

The process development created a framework that can be used to evaluate the performance of the Recurrence Score (RS) and the Set2,3 index (SET_{ER/PR} Index and Baseline Prognostic Index (BPI), collectively); alone and in combination.

### Example 2: Combination of Predicted Sensitivity to Endocrine Therapy (SET2,3 Index) and the Recurrence Score (RS) in Node-Positive Breast Cancer: Independent Validation in the SWOG Study

This example details the validation and application of independent recurrence-free interval (DRFI) and overall survival (OS) prognostic metrics from the SET2,3 index as compared with the OncotypeDX test.

### Linear Predictors of DRFI and Overall Survivor (OS) Based on SET2,3

As discussed in Example 1, the ranges of the RS, SET_{ER/PR} Index, and BPI in the PACS-01 study were 0 to 100, 0 to 3, and 0 to 4, respectively. The models developed above were then evaluated on available results of a different clinical study, the SWOG study.

FIG. 2 provides the outcome of a Kaplan-Meier Plot below stratified by RS (RS Low≤25, RS High>25) and SET2,3 (SET2,3 Low≤2.1, SET2,3 High>2.1). As demonstrated by FIG. 2, the following observations are made when evaluating the performance of the Recurrence Score (RS) and the Set2,3 index (SET_{ER/PR} Index and Baseline Prognostic Index (BPI), collectively); alone and in combination.
When SET2,3 (and hence SET_{ER/PR} Index and BPI) is small (orange and aqua lines), then SET2,3 is adding negligible if any information.
When SET2,3 (and hence SET_{ER/PR} Index and BPI) is large (blue and red lines) then SET2,3 provides significant information about the reduction of risk of a distant recurrence.
Specifically, when RS is High and SET2,3 is High, then there is a reduction in risk, which could place the individual into either the low or intermediate RS risk category. Indices based on SET2,3 (and hence SET_{ER/PR} Index and BPI) may be provided that move the individual from the RS High risk to RS Low or Intermediate Risk categories.
Similarly, individuals with RS Low and SET2,3 High also benefit with a reduced risk of a distant recurrence. However, the absolute size of this reduction is small.

### Example 3: Combination of Predicted Endocrine Activity Index (Formerly SET_{ER/PR} Index) and the Recurrence Score (RS) in Node-Positive Breast Cancer: Independent Validation in the PACS-01 Trial

This example details the development of new methodology for determining independent recurrence-free interval (DRFI) and overall survival (OS) from the SET_{ER/PR} index and Recurrence Score (RS) indexes.

### Assays:

SET_{ER/PR} index and the Recurrence Score are determined as described above. The instant examples describe the process development for methods and models revealing novel processes informing distant recurrence-free interval (DRFI) and overall survival (OS) performance from a combination of Recurrence Score (RS) and SET_{ER/PR} metrics.

RS and SET_{ER/PR} Data from Hazard Regression Analysis

The present study applied a Cox Proportional Hazards Regression Analysis to various datapoints from the PACS-01 659 samples available results for both RS and SET_{ER/PR}. The process development considered models for improved prediction of risk of distance recurrence and overall survival using combined information from OncotypeDX Recurrence Score (RS) and the SET_{ER/PR}, which does not include the BPI metrics, as described below.

### Linear Predictors of DRFI and OS Based on SET_{ER/PR} Index

Similarly, linear predictors of DRFI and OS excluding BPI are as follows:

$${Linear}_{DRFI} = 0.0 \; 113 RS - 0.54 {SET}_{ER / PR} Index \qquad EQ . 9$$

$${Linear}_{OS} = 0.0 \; 104 RS - 0.71 {SET}_{ER / PR} Index \qquad EQ . 10$$

Assuming independence of RS and SET_{ER/PR} the approximate standard errors per unit of the linear predictors Linear_{DRFI} and Linear_{OS} are approximately 0.12444 and 0.1925, respectively. The ranges of the RS and SET_{ER/PR} Index in the PACS-01 study are 0 to 100, 0 to 3, and 0 to 4, respectively. Consequently, Linear_{DRF} and Linear_{OS} range from −4.82 to 1.12 and from −5.30 to 1.11, respectively. To prevent outliers, the tails of the linear predictors are compressed as follows:

$${Compressed}_{DRFI} = \{ \begin{matrix}- 1.62 & if {Linear}_{DRFI} < - 1.62 \\ 1.13 & if {Linear}_{DRFI} > 1.13 \\ {Linear}_{DRFI} & otherwise\end{matrix} \qquad EQ . 11$$

$${Compressed}_{OS} = \{ \begin{matrix}- 5.3 & if {Linear}_{OS} < - 2.13 \\ 1.04 & if {Linear}_{OS} > 1.04 \\ {Linear}_{OS} & otherwise\end{matrix} \qquad EQ . 12$$

The predictors of DRFI and OS are subsequently rescaled from 0 to 100 as follows:

$${SET}_{DRFI} = \frac{100}{( 1.13 + 1.62 )} ( {Compressed}_{DRFI} + 1.62 ) \qquad EQ . 6$$

$${SET}_{OS} = \frac{100}{( 1.04 + 2.13 )} ( {Compressed}_{OS} + 2.13 ) \qquad EQ . 7$$

### Example 4: Determination of Breast Cancer Occurrence or Recurrence

A breast tissue sample is obtained from a subject. mRNA is extracted from the sample and analyzed for expression levels of the following genes: ESR1, PGR, BCL2, SCUBE2, Ki-67, STK15, Survivin, Cyclin B1, MYBL2, Stromelysis 3, Cathepsin L2, HER2, GRB7, SLC39A6, STC2, CA12, PDZK1, NPY1R, CD2, MAPT, QDPR, AZGP1, ABAT, ADCY1, CD3D, NAT1, MRPS30, DNAJC12, and KCNE4. The expression levels are used to calculate a recurrence score (RS) and an endocrine activity index score (EAI). These scores were then input into a Cox proportional hazard model to determine the subject's risk of breast cancer recurrence or occurrence.

### Example 5: Use of bDNA Assay for mRNA Detection

A branched DNA (bDNA) assay is used to detect mRNA expression levels in a breast tissue sample. The breast tissue sample is obtained from a subject. mRNA is extracted from the sample and analyzed for expression levels of the following genes: ESR1, PGR, BCL2, SCUBE2, Ki-67, STK15, Survivin, Cyclin B1, MYBL2, Stromelysis 3, Cathepsin L2, HER2, GRB7, SLC39A6, STC2, CA12, PDZK1, NPY1R, CD2, MAPT, QDPR, AZGP1, ABAT, ADCY1, CD3D, NAT1, MRPS30, DNAJC12, and KCNE4. The assay involved sequential hybridization of bDNA pre-amplifier, amplifier, and probe molecules labeled with a fluorescent moiety to the target mRNA. The biotin signal is then measured to quantify the mRNA expression levels.

### Example 6: Development of a Kit for Breast Cancer Risk Assessment

A kit is developed for assessing breast cancer risk. The kit included a set of nucleic acid probes, each modified with a biotin moiety or a fluorescent moiety, for determining the mRNA expression levels of 25 biomarkers selected from the group of genes listed in Tables 1 and 2. The kit also included a thermostable polymerase and deoxynucleotide triphosphates. mRNA is extracted from the sample and analyzed for expression levels of the following genes: ESR1, PGR, BCL2, SCUBE2, Ki-67, STK15, Survivin, Cyclin B1, MYBL2, Stromelysis 3, Cathepsin L2, HER2, GRB7, SLC39A6, STC2, CA12, PDZK1, NPY1R, CD2, MAPT, QDPR, AZGP1, ABAT, ADCY1, CD3D, NAT1, MRPS30, DNAJC12, and KCNE4. The reagents on the kit are combined with mRNA extracted from a breast tissue sample. The expression levels are used to calculate a recurrence score (RS) and an endocrine activity index score (EAI). These scores were then input into a Cox proportional hazard model to determine the subject's risk of breast cancer recurrence or occurrence.

While certain embodiments have been described, these embodiments have been presented by way of example only and are not intended to limit the scope of the present disclosures. Indeed, the novel methods, apparatuses, modules, instruments and systems described herein can be embodied in a variety of other forms; furthermore, various omissions, substitutions and changes in the form of the methods, apparatuses, modules, instruments and systems described herein can be made without departing from the spirit of the present disclosures. The accompanying claims and their equivalents are intended to cover such forms or modifications as would fall within the scope and spirit of the present disclosures.

What is claimed is:

1. A method for determining a risk of recurrence of breast cancer in a subject, the method comprising:
applying a hazard regression analysis to:
a recurrence score (RS) determined by a detected amount of mRNA expressed from the estrogen receptor 1 (ESR1), the progesterone receptor (PGR), BCL2, and SCUBE2 genes, whereby the mRNA is extracted from a breast tissue sample; and
an endocrine activity index score (EAI) determined by a detected amount of a plurality of genes in the group comprising SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KCNE4 genes;
whereby the mRNA is extracted from a breast tissue sample.

2. The method of claim 1, wherein determining the recurrence score further comprises determining a detected amount of mRNA expressed from a set of proliferation genes comprising one or more of Ki-67, STK₁₅, Survivin, Cyclin B₁, and MYBL₂ genes.

3. The method of claim 1, wherein determining the recurrence score further comprises determining a detected amount of mRNA expressed from a set of invasion genes comprising one or more of Stromelysis 3 and Cathepsin L₂ genes.

4. The method of claim 1, wherein determining the recurrence score further comprises determining a detected amount of mRNA expressed from a set of human epidermal growth factor receptor genes comprising one or more of HER₂ and GRB₇.

5. The method of claim 1, wherein determining the recurrence score further comprises determining a detected amount of mRNA expressed from a set of reference genes comprising one or more of Beta-actin, GAPDH, RPLPO, GUS, and TFRC.

6. The method of claim 5, wherein the detected amount of mRNA expressed from the ESR1, PGR, BCL2, and SCUBE2 genes is normalized to one or more reference genes in the set of reference genes.

7. The method of claim 1, wherein determining the endocrine activity index score is determined by a detected amount of the ten genes in the group consisting of MAPT, CD2, CD3D, QDPR, PDZK1, STC2, MRPS30, DNAJC12, SCUBE2, and KCNE4 genes.

8. The method of claim 1, wherein determining the endocrine activity index score is determined by a detected amount of the ten genes in the group consisting of SLC39A6, STC2, CA12, ESR1, PDZK1, NPY1R, CD2, MAPT, QDPR, and AZGP1 genes.

9. The method of claim 1, wherein determining the endocrine activity index score is determined by a detected amount of the ten genes in the group consisting of QDPR, AZGP1, ABAT, ADCY1, CD3D, NAT1, MRPS30, DNAJC12, SCUBE2, and KCNE4 genes.

10. The method of claim 1, wherein determining the endocrine activity index score further comprises determining a detected amount of mRNA expressed from a set of reference genes comprising one or more of AK2, APPBP2, ATP5J2, DARS, LDHA, UBE2Z, UGP2, VDAC2, and WIPF2.

11. The method of claim 1, wherein the endocrine activity index score (EAI) is determined by
$$EAI = \frac{{\sum }_{i = 1}^{18} Ti}{i} - \frac{{\sum }_{j = 1}^{10} Rj}{j} + 2 .$$

12. The method of claim 1, wherein the recurrence score is determined by a detected amount of mRNA expressed from the ESR1, PGR, BCL2 gene, and SCUBE2 genes inputted into the formula:
13.424+5.420*(nuclear grade)+5.538*(mitotic count)−0.045*(ER H-score)−0.030*(PR H-score)+9.486*(0 for negative/equivocal and 1 for HER2 positive).

13. The method of claim 1, wherein the hazard regression model is a Cox proportional hazard model for distant recurrence-free interval (DRFI).

14. The method of claim 12, wherein the Cox proportional hazard model is
$$h ( t ) ≅ {h}_{0} ( t ) exp ( {β}_{1} RS + {β}_{2} EAI Index + {β}_{3} BPI ) \qquad EQ . 1$$
where β₁, β₂, and β₃, are the (natural) logarithms of the hazard rates for RS, EAI and BPI, respectively, and h₀(t) is the baseline hazard function.

15. The method of claim 1, wherein the distant recurrence-free interval (DRFI) is determined by:
$${Linear}_{DRFI} = 0.0113 RS - 0 \text{.54} {SET}_{\frac{ER}{PR}} - 0.8 BPI \qquad EQ . 2$$
$$or$$
$${Linear}_{DRFI} = 0.0 \; 113 RS - 0.54 {SET}_{\frac{ER}{PR}} Index \qquad EQ . 9$$

16. The method of claim 14, the distant recurrence-free interval (DRFI) is provided on a scale from 0 to 100 determined by:
$${Compressed}_{DRFI} = \{ \begin{matrix}- 4.8 & if {Linear}_{DRFI} < - 4.8 \\ 1.1 & if {Linear}_{DRFI} > 1.1 \\ {Linear}_{DRFI} & otherwise\end{matrix} \qquad EQ . 4$$
$$\text{and/or}$$
$${SET}_{DRFI} = \frac{100}{( 1.10 + 4.80 )} ( {Compressed}_{DRFI} + 4.80 ) \qquad EQ . 6$$
$$or$$
$${Compressed}_{DRFI} = \{ \begin{matrix}- 1.62 & if {Linear}_{DRFI} < - 1.62 \\ 1.13 & if {Linear}_{DRFI} > 1.13 \\ {Linear}_{DRFI} & otherwise\end{matrix}$$
$${SET}_{DRFI} = \frac{100}{( 1.13 + 1.62 )} ( {Compressed}_{DRFI} + 1.62 )$$

17. The method of any one of claims 1-13, wherein an overall survival (OS) of the subject is determined by:
$${Linear}_{OS} = 0.0 \; 104 RS - 0 \text{.71} {SET}_{\frac{ER}{PR}} - 0.80 BPI \qquad EQ . 3$$
$$or$$
$${Linear}_{OS} = 0.0 \; 104 RS - 0 \text{.71} {SET}_{\frac{ER}{PR}} Index . \qquad EQ . 10$$

18. The method of claim 16, wherein the overall survival (OS) is provided on a scale from 0 to 100 determined by:
$${Compressed}_{OS} = \{ \begin{matrix}- 5.3 & if {Linear}_{OS} < - 5.3 \\ 1.1 & if {Linear}_{OS} > 1.1 \\ {Linear}_{OS} & otherwise\end{matrix} \qquad EQ . 5$$
$${SET}_{OS} = \frac{100}{( 1.1 + 5.3 )} ( {Compressed}_{OS} + 5.3 ) \qquad EQ . 7$$
$$or$$
$${Compressed}_{OS} = \{ \begin{matrix}- 5.3 & if {Linear}_{OS} < - 2.13 \\ 1.04 & if {Linear}_{OS} > 1.04 \\ {Linear}_{OS} & otherwise\end{matrix} \qquad EQ . 12$$
$${SET}_{OS} = \frac{100}{( 1.04 + 2.13 )} ( {Compressed}_{OS} + 2.13 ) \qquad EQ . 7$$

19. The method of claim 1, wherein the mRNA expression levels are determined with a set of probe pairs each modified with a detectable moiety.

20. The method of claim 18, wherein the detectable moiety is a biotin moiety.

21. The method of claim 18, wherein the detectable moiety is a fluorescent moiety.

22. The method claim 1, wherein the mRNA expression levels are determined via hybridization of one or more probes complementary to the target mRNA genes in a branched DNA (bDNA) assay.

23. The method claim 1, wherein the bDNA assay comprises sequential hybridization of the bDNA pre-amplifier, amplifier, and probe molecules labeled with a detectable moiety to the target mRNA.

24. The method of any of claim 1, wherein the hazard regression analysis provides a
patient specific score for the subject.

25. The method of claim 1, wherein the hazard regression analysis provides a population specific score for the subject.

26-58. (canceled)
